(19) United States
(12) Patent Application Publication
(10) Pub. No.: US 2010/0291620 A1
(43) Pub. Date: Nov. 18, 2010

(54) BIO-FUEL PRODUCTION SYSTEM

(76) Inventors: James P. Abrams, Dallas, PA (US); William P. Abrams, Dallas, PA (US)

Correspondence Address: MORGAN, LEWIS & BOCKIUS LLP, 1701 MARKET STREET, PHILADELPHIA, PA, 19103-2921, US

(21) Appl. No.: 12/443,395

(22) PCT Filed: Sep. 28, 2007
(86) PCT No.: PCT/US2007/021043

§ 371 Date: May 29, 2009

Related U.S. Application Data
(60) Provisional application No. 60/848,559, filed on Sep. 29, 2006.

(51) Int. Cl. C12P 1/00; C12M 1/00

(52) U.S. Cl. 435/41; 435/289.1; 435/303.1

(57) ABSTRACT

A biological material production system having an enclosure; a plant advancer located within th enclosure, the plant advancer configured to move one or more plants from a first position to a second position in a predetermined path; and at least one harvester positionable proximate the second position and configured to remove at least a portion of the one or more plants from the plant advancer. A biofuel production system having a biological material production system a harvesting device configured to harvest the biological material as the biological material matures, at least a portion of the harvesting system being substantially within the enclosure, the harvesting system configured to extract liquid from the biological material; a fermenting device proximate the harvesting system and configured to accept and ferment the liquid from the harvesting system; and a distillation device proximate the fermenting device and configured to distill the fermented liquid.

### CROSS-REFERENCE TO RELATED APPLICATION

This application claims priority to U.S. Provisional Application No. 60/848,559 filed Sep. 29, 2006, the entire contents of which are hereby incorporated by reference herein.

### INCORPORATION BY REFERENCE

All references cited herein are hereby incorporated by reference as if set forth in their entirety herein.

### BACKGROUND

The present invention relates to bio-fuel production. In some systems bio-fuel is derived from crops. A common crop for use in bio-fuel is corn. Other systems exist for harvesting crops which produce high sugar juices, such as sweet sorghum, sugar cane and others. In some systems, the juice is pressed out of the stalks of the crops and distilled to produce fuel-grade ethanol. Such methods are discussed in U.S. Pat. No. 4,613,339 Gunnerman, et. al., which is incorporate by reference in its entirety herein.

Among the disadvantages of these methods is the difficulty and time delay associated with transporting harvested crops to a bio-fuel production facility. With a weight of 40 tons per acre, transportation of harvested sweet sorghum stalks to a central facility for pressing and distillation or fermentation is difficult and costly. Another possible disadvantage results from the speed with which sugars in the stalk begin to sour. In some processes fermentation must begin rapidly (e.g., within 30 minutes to 40 minutes of harvest) in order to prevent bacterial contamination and high temperatures (e.g., temperatures in excess of 60 degrees Fahrenheit for some plants) from converting sugar in the stalk to lactic acid. This may be problematic, for example in connection with Sweet Sorghum as a feed stock, where the presence of lactic acid inhibits yeast from fermenting sugars to ethanol.

Also one of the challenges with known systems involving crops that must be processed rapidly occurs when crops become ready to harvest all at the same time.

In addition, there are additional reactions which reduce the amount of fermentable material in the juice which are time-dependent. For example, naturally occurring protease or wild yeast may contaminate juice when exposed in a natural or uncontrolled environment.

Therefore, methods and apparatuses are also known for the field-pressing of juice from harvested stalks. However, juice which is field-pressed from some high sugar crops must be immediately processed in order to create an appreciable amount of ethanol and limit exposure to outside bacteria and temperature in the environment. In some cases, such as with C4 grasses or Sweet Sorghum, this is because juices produced from the stalk begin to sour when the stalk wall is ruptured or crushed.

Also, in conventional farming, where most of the crop matures at the same time, a large amount of harvesting equipment is required for a short period of time; an inefficient use of equipment resources.

One such method, known as the “Piedmont harvest system” (see, e.g., Rains, G. C., J. S. Cundiff, and G. E. Welbaum, Sweet Sorghum for a Piedmont Ethanol Industry, in New Crops, pp. 394-399 (J. Janick and J. E. Simon eds., Wiley, 1993 which is hereby incorporated by reference in its entirety herein) requires a whole-stalk harvester, loaders, trailers and truck-mounted screw presses located inside a bunk silo in order to minimize the time from harvest to the onset of fermentation.

Currently there is a need for a more efficient system for the production of biofuel and in particular ethanol.

### SUMMARY OF THE INVENTION

In one embodiment there is disclosed a biofuel production system including an enclosure; a plant advancer located within the enclosure, the plant advancer configured to move one or more plants from a first position to a second position; and at least one harvester positionable proximate the second position and configured to remove at least a portion of the one or more plants from the plant advancer. In a further embodiment of the biofuel production system the first position is associated with an immature plant and the second position is associated with a mature plant. Another embodiment of the biofuel production system includes a crusher proximate the at least one harvester and configured to separate juices from the at least a portion of the one or more plants; and a biofuel processing device proximate the at least one harvester and configured to process the separated juices into biofuel. In another embodiment of the biofuel production system the at least one harvester includes the crusher. In yet another embodiment of the biofuel production system the one or more plants are grown hydroponically. In still another embodiment of the biofuel production system the one or more plants are grown aeroponically. In another embodiment of the biofuel production system the plant advancer further comprises a controller configured to move the one or more plants continually from the first position to the second position as the one or more plants mature. In a further embodiment of the biofuel production system, the plant advancer is configured to include vertically stacked plants. In another embodiment of the biofuel production system the plant advancer is configured in a pyramid configuration. In a further embodiment of the biofuel production system the plant advancer includes a plurality of troughs stacked on the plant advancer in an offset vertical configuration, each trough accommodating a plurality of plants. In a further embodiment of the biofuel production system the plant advancer is arranged in a row with at least one additional plant advancer. In a further embodiment of the biofuel production system the plant advancer is moveable from the first position to the second position. In one embodiment, the biofuel production system also includes an advancer guide configured to guide the plant advancers from the first position to the second position. In a further embodiment of the biofuel production system biofuel processing device also includes a fermentation device configured to produce fermented juice from the separated juice and a distillation device configured to refine the fermented juice into biofuel. In a further embodiment of the biofuel production system the crusher expresses bagasse and the biofuel production system further comprises a combustion device proximate the at least one harvester that is configured to combust the bagasse and to produce for recycling in the biofuel production system at least one of heat and power. In a further embodiment of the biofuel production system the one or more plants include sweet sorghum and the biofuel includes ethanol. In a further embodiment of the biofuel production system the plant advancer includes a plurality of adjacent substantially parallel rows of plant holders wherein each of the rows has a first end corresponding to the first position and a second end corresponding to the second position and wherein the first end of each row is proximate the second end of an adjacent row. In one embodiment, the biofuel production system also includes at least one light source configured to illuminate at least a portion of the one or more plants. In a further embodiment of the biofuel production system the at least one light source comprises an LED.

There is also disclosed a biofuel production system having an enclosure configured to control an environment; a biological material production device substantially within the enclosure configured for the continual production of biological material; a harvesting device configured to harvest the biological material as the biological material matures, at least a portion of the harvesting system being substantially within the enclosure, the harvesting system configured to extract liquid from the biological material; a fermenting device proximate the harvesting system and configured to accept and ferment the liquid from the harvesting system; and a distillation device proximate the fermenting device and configured to distill the fermented liquid. In one embodiment of the biofuel production system the harvesting device produces bagasse and the biofuel production system further comprises a heat return system configured to process the bagasse to produce heat and to deliver the heat to an area proximate the biological material production device. One embodiment of the biofuel production system also includes a power generator configured to convert the produced heat to power; and a power return system configured to return at least a portion of the power produced by the power generator to the biofuel production system. In one embodiment of the biofuel production system the biological material production device is configured to grow biological material hydroponically. In one embodiment of the biofuel production system the biological material production device is configured to grow biological material aeroponically. In one embodiment of the biofuel production system the biological material includes C4 grass plants. In one embodiment of the biofuel production system the C4 grass plant include Sweet Sorghum. In one embodiment of the biofuel production system the biological material production device comprises a biological material configuration that results in a biological material yield of greater than approximately one million plants per acre per year.

There is also disclosed a biofuel production system having a plant bed having at least one plant advancer; a plant support moveably attached to the at least one plant advancer; a plurality of planter pots supported by the plant support, each planter pot containing at least one plant; a harvester positioned at a harvesting side of the plant bed, wherein the at least one plant advancer is configured to continuously advance the plants toward the harvesting side of the plant bed and wherein the harvester is configured to harvest the plants at the harvesting side and extract liquid from the harvested plants; and a continuous fermentation device proximate the harvester and configured to ferment the liquid into biofuel. In one embodiment of the automated continuous biofuel production system the biofuel comprises ethanol. In one embodiment of the biofuel production system the plants include C4 grass plants. In one embodiment of the biofuel production system the plurality of planter pots are supported by the plant support in a vertical configuration. In one embodiment of the biofuel production system the at least one plant advancer includes a plurality of plant advancers configured in substantially parallel rows. In one embodiment of the biofuel production system the harvester includes a cutting device configured to cut the plants; a feeding section configured to catch the cut plants; a sprayer configured to spray liquid on the cut plants; a crusher configured to extract liquid from the cut plants; and a liquid collection device configured to collect the extracted liquid. In one embodiment of the biofuel production the planter pots are enclosed within an enclosure. In one embodiment of the biofuel production system the continuous fermentation device produces carbon dioxide; the automated continuous biofuel production system further comprising a carbon dioxide conduit configured to deliver at least a portion of the produced carbon dioxide into the enclosure. In one embodiment of the biofuel production system the at least one plant advancer includes adjacent plant advancers that advance the plants in opposite directions. In one embodiment, the biofuel production system also includes a combustion unit proximate the harvester, configured to combust bagasse produced from the harvester and to produce energy for use by the automated continuous biofuel production system. In one embodiment, the biofuel production system also includes a gasification unit operating in at least one of a Fischer-Tropsch or a pyrolysis process, the gasification unit being proximate the harvester and configured to gasify bagasse produced from the harvester and to produce heat and biofuel. In one embodiment of the biofuel production system the combustion unit is located at least partially within an enclosure and is configured to produce heat used in the operation of the automated continuous biofuel production system.

There is also disclosed a substantially self-contained continuous ethanol production system includes a plurality of plant advancers arranged in a plurality of rows, the plant advancers having a plurality of sweet sorghum plants and being configured to move the plurality of sweet sorghum plants to an end position as the sweet sorghum plants mature; a processor positionable at the end position, the processor including (i) a cutting device configured to cut the sweet sorghum plants; (ii) a feeding section configured to catch the cut sweet sorghum plants; (iii) a sprayer configured to spray the cut sweet sorghum plants; (iv) a crusher configured to extract juice from the sweet sorghum plants; and (v) a juice collection device configured to collect the juice; a container coupled to the juice collection device configured to receive, hold and ferment the collected juice; a distillation device configured to separate ethanol from the fermented juice; and a combustion device proximate the distillation device and proximate the plurality of plant advancers, the combustion device being configured to produce heat and energy useable by the ethanol production system through the combustion of bagasse generated by the processor.

There is also disclosed a decentralized ethanol production system including a plurality of biological material production sites each site having a biological material production apparatus configured to continually produce sweet sorghum; a harvesting device configured to continually harvest the sweet sorghum; and an ethanol production apparatus configured to continually produce ethanol from the harvested sweet sorghum. In one embodiment, the decentralized ethanol production system also includes a by-product consumption system configured to produce energy for use by the decentralized ethanol production system wherein the by-product consumption system includes a by-product combustion device.

There is also disclosed a biological material production system that includes an enclosure; an plant advancer located within the enclosure, the plant advancer configured to move one or more plants from a first position to a second position in a predetermined path; and at least one harvester positionable proximate the second position and configured to remove at least a portion of the one or more plants from the plant advancer. In one embodiment of the biological material production system the one or more plants are configured in a vertical planting configuration having a plurality of plant levels. In one embodiment of the biological material production system the at least one harvester includes a robotic arm having a plurality of cutting devices configured to remove the at least a portion of the one or more plants from at least two of the plant levels simultaneously. In one embodiment of the biological material production system the plant advancer moves the one or more plants continuously. In one embodiment of the biological material production system the continuous movement of the plants is intermittent.

### BRIEF DESCRIPTION OF THE FIGURES

FIG. 1 is a block diagram of system 1000 according to an embodiment of the present invention.

FIG. 2 is a plan view of a biofuel production system according to an embodiment of the present invention.

FIG. 3 is an enlarged view of one embodiment of a plant bed of the system shown in FIG. 2.

FIG. 4 is an enlarged view of the harvester, processor device (HPD) of the system shown in FIG. 2.

FIG. 5 is an enlarged view of a second embodiment of the HPD of the system shown in FIG. 2.

FIG. 6 is an enlarged view of an embodiment of a plant bed of the system shown in FIG. 2.

FIG. 7 is an alternative embodiment of plant beds according to an embodiment of the present invention.

FIG. 8 is a perspective view of a vertical planting system according to one embodiment of the present invention.

FIG. 9 is a plan view of one embodiment of a vertical planting system according to one embodiment of the present invention.

FIG. 10 is a cross sectional view of the vertical planting system of FIG. 9.

FIG. 11 is a cross sectional view of the vertical planting system of FIG. 9.

FIG. 12 is an illustration of a trough according to one embodiment of the present invention.

FIG. 13 is an illustration of a trough according to one embodiment of the present invention.

FIG. 14 is an illustration of a trough according to one embodiment of the present invention.

FIG. 15 is an illustration of a cross sectional view of a vertical planting system according to one embodiment of the present invention.

### DETAILED DESCRIPTION

One aspect of an embodiment of the present invention provides s a system for the economical, energy-efficient production of bio-fuel (e.g., ethanol). Another aspect of an embodiment of the present invention provides a system which maximizes the amount of biofuel produced. Another aspect of an embodiment of the present invention provides a biofuel production system which produces biofuel on a continuous, year-round basis. Another aspect of an embodiment of the present invention provides a system and/or method for constant output of biofuel, independent of weather and climate. Still another aspect of an embodiment of the present invention provides a system and/or method to produce high sugar content feedstock for the production of biofuel independent of weather and climate. Another aspect of an embodiment of the present invention minimizes the amount of water required to create biofuel. In one embodiment, at least a portion of the water used to supply nutrients to plants is recycled back through the system. In one embodiment, the plants used to create ethanol (e.g., Sweet Sorghum) do not require boiling to produce adequate carbohydrate rich juices. Another aspect of an embodiment of the present invention includes the manufacture of biofuel on land that is not currently suited for agricultural purposes. Another aspect of one embodiment of the invention includes the year round production of plants and ethanol in a single location. Another aspect of one embodiment of the present invention includes increasing the output of sweet sorghum for ethanol production by increasing the density of sweet sorghum grown in a given site that is proximate an ethanol production facility. Still another aspect of one embodiment of the present invention accommodates the decentralization of biofuel production in that multiple self-contained biofuel production systems where plant growth, harvesting and ethanol production occur at the same site avoid the necessity for large scale central ethanol production facilities remotely located from multiple harvesting locations.

Overall System

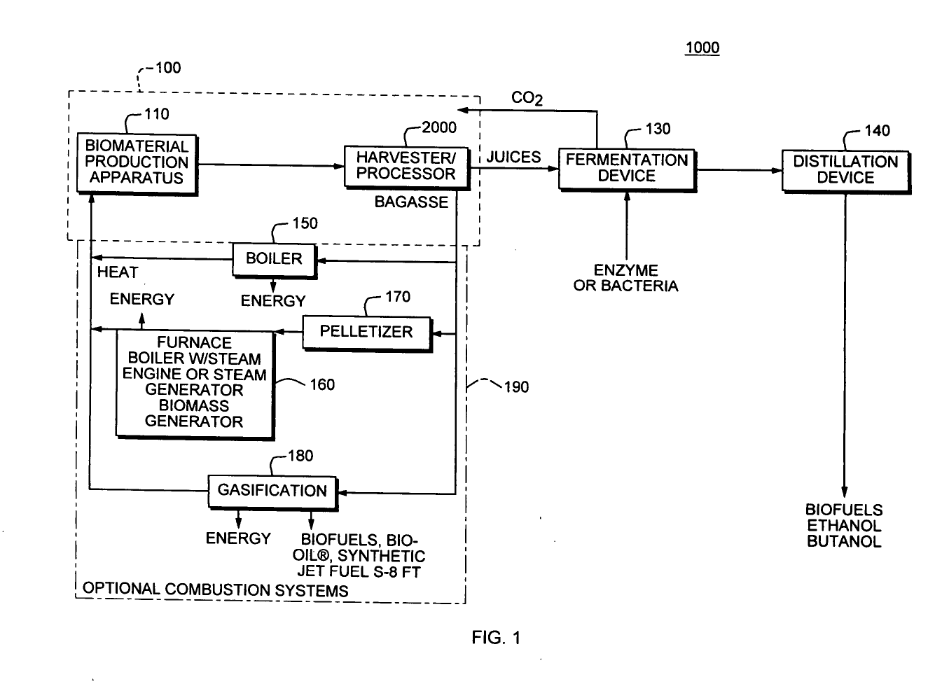

One embodiment of the invention is an at least partially automated, continuous, closed-loop biofuel production system. In one embodiment, illustrated in FIG. 1, system 1000 is a substantially self-sustaining biofuel production system. In one embodiment of the biofuel production system 1000 includes a biological material production device 110, a harvesting/production device (HPD) 2000, a fermentation device 130, and a distillation device 140. In one embodiment, biofuel production system 1000 also includes one or more heat/energy production devices 190. In one embodiment, biofuel production system 1000 is at least partially enclosed within an enclosure (e.g., greenhouse 100).

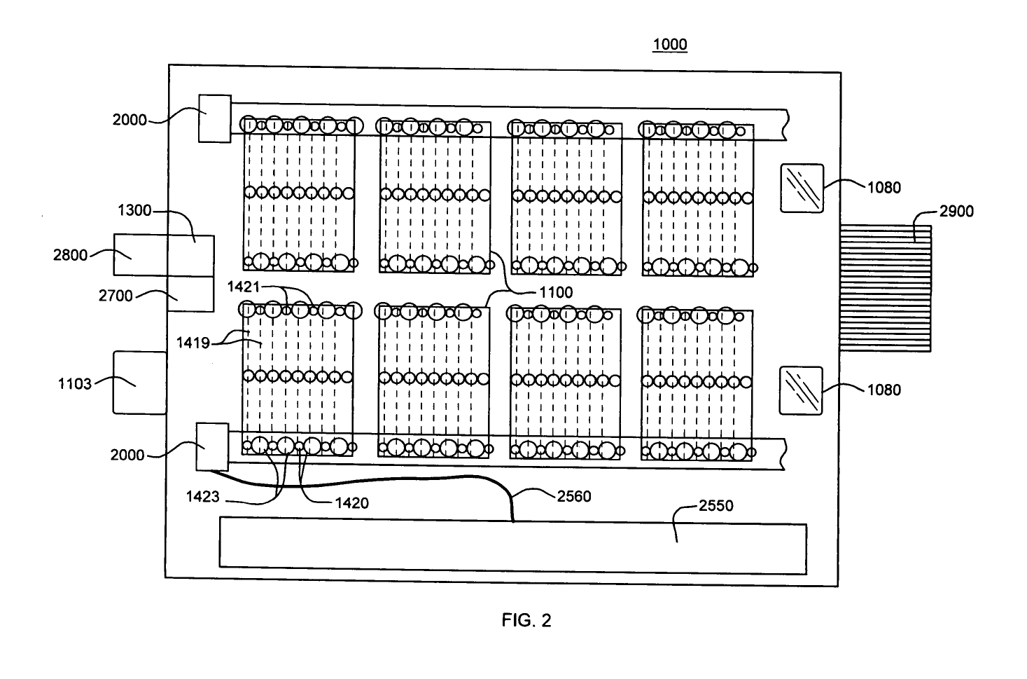

In one embodiment, the biological material production device is configured for the continual production of biological material. In one embodiment, the HPD 2000 is configured to continually harvest the biological material as the biological material as the biological material matures. In one embodiment, at least a portion of the HPD 2000 is located in greenhouse 100. The HPD 2000 is further configured to collect juices from the biological material. In a preferred embodiment, juices are fermented in fermentation device 130 which is located proximate HPD 2000. As further illustrated in FIG. 2, fermented juices are conveyed from the fermentation device 130 to distillation device 140 where the fermented fuels are distilled into a desired biofuel product.

In one embodiment, by-products of system 1000 are collected for re-use by system 1000. Those by-products include heat, carbon dioxide and bagasse. In one embodiment, by-products produced by any portion of system 1000 are collected and re-used by system 1000. In For example, in one embodiment, carbon dioxide (CO₂) is introduced to system 1000 to promote biological growth. For example, in one embodiment, fermentation device 130 produces carbon dioxide (CO₂) that is recycled back to greenhouse 100 to enhance biological material growth.

In some embodiment, bagasse produced from the HPD device is also collected. In a preferred embodiment, bagasse is treated to produce heat that is returned to system (e.g., to a location proximate the plants 1420, to biological material production device 110, to greenhouse 100, and the distillation unit 140 to produce ethanol or anywhere the application of heat might be beneficial to the system) to promote biological material growth and/or produce energy used to operate the biofuel production system 1000 (discussed in more detail below).

Thus, in a preferred embodiment, biofuel production system is a closed loop system such that the by-products of the biofuel production system (e.g., CO₂, and bagasse) are at least partially recycled back to the system.

In one embodiment, the system is automated and the temperature, water, chemistry, plant nutrition, and photosynthesis and the like may be optimized producing far more plants per acre, in any weather, as compared with prior art systems. In a preferred embodiment of the present invention, system 1000 produces at least approximately 60,000 gallons of ethanol per acre per year. One embodiment of the present invention consumes approximately 3 million plants per acre per year.

The preferred embodiment of system 1000 is substantially self-contained, and thus may be situated in arid locations such as deserts, or any environmentally unsuitable parcel of land where the soil has very little organic content or where there are environmental hazards such as coal fields or landfills.

The present invention may be embodied as an automated, continuous ethanol production system 1000 that includes a plurality of rows 1410 of plant advancer devices 1419 each having a plurality of plants 1420 capable of creating sugar containing juices 1505 when they mature, for moving the plants 1420 along rows 1410 as they mature to an end location, a harvester, processor device (HPD) 2000 capable of moving to the end location of each row 1410. In one embodiment, the HPD 2000 includes a cutting device 2100 for cutting the plants 1420, a feeding section 2200 for catching the cut plants 1420, a sprayer 2250 for spraying liquids on the cut plants 1423, a crusher 2300 to extract sugar containing juices 1505 from plants 1420, a liquid collection device 2400 for collecting the juices 1505, a container 2500 coupled to the liquid collection device for receiving the collected juices 1505 and for holding and fermenting the juices 1505 into ethanol. In one embodiment, biofuel production system 1000 also includes a distillation unit 140 for concentrating the ethanol.

In another embodiment, there is an automated, continuous ethanol production system 1000. In one embodiment, ethanol production system includes plants 1420 planted in moveable pots 1415. The pots 1415 are moved along a row 1410 as they mature toward a harvester, processor device (HPD) 2000. In one embodiment, the HPD 2000 employs a cutting device 2100 for cutting the mature plants 1423, a feeding section 2200 for catching the cut plants 1423, a sprayer 2250 for spraying liquids on the cut plants 1423. The cut plants 1423 are then fed to a crusher 2300 to separate sugar containing juices 1505 from waste bagasse 1507. In one embodiment, the bagasse 1507 is used as a fuel for further processing, while the juices 1505 are collected in a container 2500 for fermentation and distillation. The system 1000 rapidly harvests the juices 1505 to minimize its deterioration and maximize ethanol production. In one embodiment, the system geometry allows for constant maturation of plants 1423, a constant supply of juices 1505 and constant production of ethanol. The geometries also allow for maximum production per square foot of space. An alternative embodiment employs a multiple level system.

Various embodiments to achieve a desired density of biological material are within the scope of the present invention. In one embodiment, ethanol production system 1000 includes rows 1410. In one embodiment rows 1410 are generally parallel straight rows. In one embodiment, plants 1420 in adjacent rows move in opposite directions thereby maximizing space usage. In one embodiment, ethanol production system 1000 includes rows 1410 that are generally parallel straight rows.

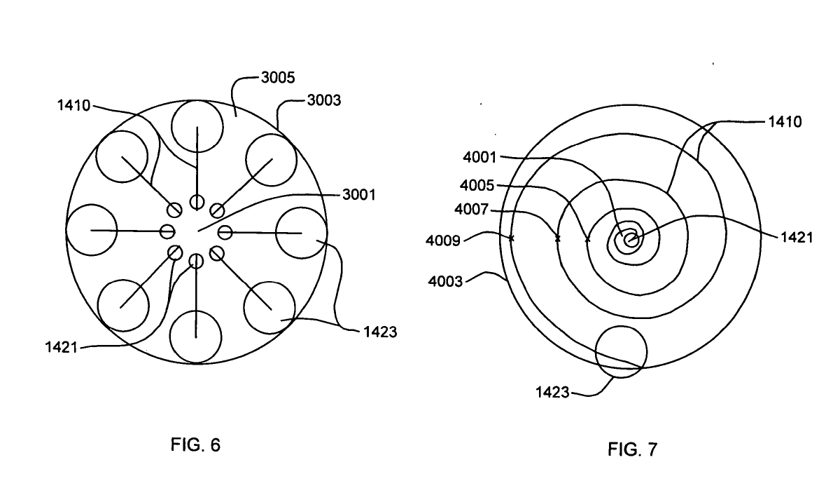

In one embodiment, ethanol production system 1000 includes rows 1410 that radiate from a central location 3001 (e.g., FIG. 6) on a generally circular bed 3005 and the seedling plants 1421 move radially outwardly toward a perimeter 3003 maximizing space usage.

In another embodiment, (e.g., illustrated in FIG. 7) ethanol production system 1000 includes rows 1410 that are generally spiral shaped rows starting at a central location 4001 on a generally circular bed 1100, and the plants 1420 move circularly outwardly toward a perimeter 4003, and is designed such that the spacing between adjacent turns increases toward the perimeter, thereby maximizing space usage.

In another embodiment, ethanol production system 1000 includes plant advancers 1419 that are arranged in a matrix. In one embodiment, plant advancers move from the matrix when the plants have matured to the point that they are ready for harvesting.

In another embodiment, multiple level planters are used hanging from a support system, thereby allowing a greater output per square foot.

System components and features of various embodiments will now be described in more detail.

Biological Material

In one embodiment biological material useful in the present invention includes any biological material that can be processed (e.g., fermented and/or distilled) in the system of the present invention to convert the biological material into biofuel such as ethanol. In one embodiment, the biological material is a feed stock crop. In one embodiment, biological material includes material taken from the Poaceae/Gramineae family of plants or C4 grass family of plants. In one embodiment, the biological material, includes material from C4 grass plants. The biological material may include such C4 grass plants that include one or more of the following subfamilies: Arundinoideae, Bambusoideae, Centothecoideae, Chloridoideae, Panicoideae, Pooideae and Stipoideae. In one embodiment, biological material includes material that produces carbohydrates and/or fiber. In a preferred embodiment, biological material includes switch grasses. Preferably, biological material includes Sorghum bicolor, also known as Sweet Sorghum. In one embodiment, the variety of Sweet Sorghum used is at least one of the M81E, Dale, Kellar, Topper 76-6, Delta, and Theis varieties. In a preferred system, biological material is processed to produce juice (e.g., having fermentable carbohydrates) and fiber (e.g., bagasse that may be burned to produce heat and energy that is returned to the system). In one embodiment, biological material such as sweet sorghum is the preferred biological material because it produces readily fermentable carbohydrate in its stalk which is readily accessible using the system and methods of the present invention. Also, biological material such as sweet sorghum is preferred because its stalks grow in a geometry that is particularly useful in cultivation systems that promote a high density vertical plant bed. In some embodiments, Sweet Sorghum is selected because it has a relatively short time to maturity. Also, in some embodiments, ethanol production systems using sweet sorghum provide useful by-products including, without limitation, grain agricultural feed from seed for human and livestock consumption and fiber for paper/building materials.

Controlled Environment Agriculture

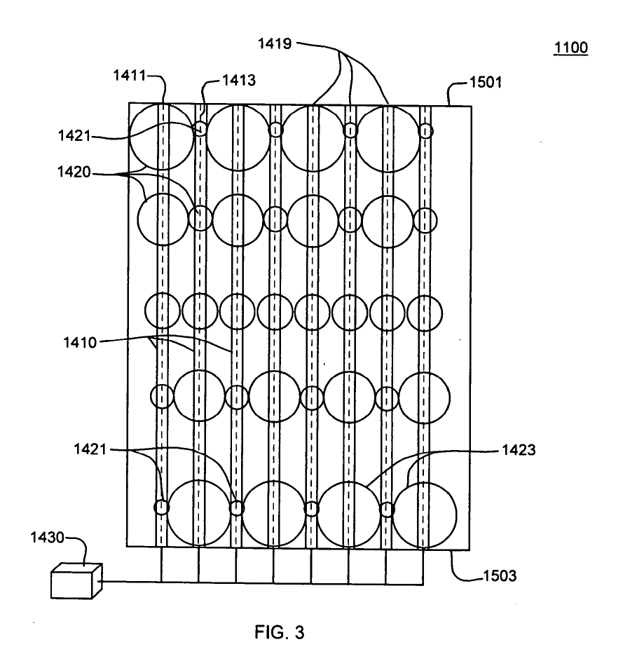

In a preferred embodiment, biological material of the present invention is grown in a controlled environment such as greenhouse 100 (one embodiment of which is illustrated in FIG. 3). In a preferred embodiment, greenhouse 100 is configured to accommodate a high density of biological material horizontally and/or vertically.

In one embodiment, greenhouse 100 includes a soil-less system. In one embodiment, greenhouse 100 includes a hydroponic greenhouse. In one embodiment, greenhouse 100 includes an aeroponic greenhouse.

In one embodiment, system 1000 employs chemical mixing units and equipment required for hydroponic or aeroponic farming. System 1000 may also use known automated farming, greenhouse and other techniques which are assumed to be known and not described in detail here.

In one embodiment, greenhouse 100 is located proximate the harvesting, distillation and/or fermentation devices to promote the rapid conversion of biological material grown within greenhouse 100 into biofuel such as ethanol. In one embodiment, some or all of the harvesting, distillation and/or fermentation equipment are located within greenhouse 100.

In one embodiment, system 1000 includes biological material production devices (BMPD) 110 (FIG. 2). In one embodiment, BMPD 110 is located within greenhouse 100. In one embodiment BMPD 110 includes a plurality of plant beds 1100 (such as illustrated in FIG. 3). Plant beds 1100 are described in more detail below.

In one embodiment, system 1000 employs a heater 1300 for controlling the temperature of greenhouse 100. In one embodiment, at least a portion of heater 1300 is located within greenhouse 100. In one embodiment, heater 1300 burns any of a number of various fuels including coal and the biological material grown in the greenhouse. Therefore, this may be well adapted for use in areas having slag coal available. Such areas are typically depleted of organic matter and do not grow vegetation well.

System 1000 may also include a germination section 2900. In one embodiment, germination section 2900 is attached to greenhouse 100. In one embodiment, germination section 2900 is located within greenhouse 100. In one embodiment, germination section 2900 is configured to create an optimum environment for producing seedlings 1421 from seeds. In one embodiment, germination section 2900 is controlled for humidity, temperature and light. In one embodiment, germination section 2900 is adapted to include multiple stacked levels since seedlings lack the height and width of mature plants. In one embodiment, seedlings in germination section 2900 are between approximately four inches and approximately six inches in height.

Referring back to FIG. 2, the system employs known technology to pump liquids in tanks 1103 throughout the system. In one embodiment, the system is capable of recovering rainwater using known techniques to provide a water source for use in the system.

Continuous Growth

One embodiment of system 1000 promotes the continuous growth of biomaterials. In one embodiment, system 1000 is configured to accept the continual introduction of seedlings to plant beds 1100 as mature plants 1423 are harvested from plant beds 1100. Thus, in one embodiment there is preferably a constant supply of mature biological material to be harvested for the production of biofuel. In one embodiment, the continuous supply of mature biological material throughout the year can be harvested by much less equipment than that which is required for harvesting a similar annual quantity of crops grown in a traditional farm.

In one embodiment, system 1000 includes plant advancer 1419 (see e.g., FIGS. 2, 3, 7) preferably located within greenhouse 100. In one embodiment, plant advancer 1419 is driven by a belt, chain or other actuator controlled by a controller 1430. Some embodiments of plant advancer 1419 are described in more detail below. In one embodiment, plant advancer 1419 is configured to move one or more plants from a first position to a second position. In one embodiment, the first position is associated with less mature plants and the second position is associated with a more mature plants. In one embodiment when the plant is in the first position it is a seedling and when the plant is in the second positions it is ready for harvesting. In one embodiment, plants are moved from the first position to a second position continuously. In one embodiment, the continuous movement of plants is uninterrupted. In another embodiment, the continuous movement of plants from the first position to the second position is intermittent. In one embodiment, the intermittent movement of plants includes movement at a pre-determined interval. In one embodiment, the pre-determined interval is a uniform interval. In another embodiment, the pre-determined interval varies over time. In one embodiment, the rate and interval of movement is determined based upon the expected rate the plants will mature and the logistics of operating the biomaterial production apparatus.

In one embodiment, plant advancer 1419 is controlled by controller 1430. In one embodiment, controller 1430 is selected from those controllers known in the art and advances plants at a substantially continuous rate from the first position to the second position. In one embodiment, plant advancer 1419 cooperates with plant bed 1100. In one embodiment, plant advancers 1419 include a linear actuator such as those sold by Nook Industries, Inc. of Cleveland, Ohio. In one embodiment, plant advancer may also include floor conveyors (e.g., belt driven or chain driven) such as those sold by Velmac, Inc. of Fenton, Mo.

In one embodiment, plants 1420 are held on plant advancer 1419 by the stem or roots of plant 1420. Plant advancer 1419 may also hold a number of pots containing the plants 1420. In one such embodiment, controller 1430 causes plant advancer 1419 to move the pots along the row 1410 in a predetermined manner. In one embodiment, the pots are soil pots. In another embodiment, the pots are soil-less pots configured to accommodate hydroponic or aeroponic farming techniques.

In one embodiment, the plants are in a pot-less structure held in place by plant advancer 1419. In one embodiment, nutrient rich water passed by the roots of the pot-less system. In another embodiment, plant advancer 1419 drags plant roots through a nutrient right water. The plant advancers 1419 may also be configured to include an elevation grade such that plants 1420 descend by gravity as they progress along the length of the rows 1410. In one embodiment, this elevation grade facilitates the movement of plants toward a harvester.

In one embodiment, illustrated in FIG. 3, plant bed 1100 has a plurality of rows 1410 of plants 1420. One embodiment of plant bed 1100 is shown in an enlarged plan view in FIG. 3. Here rows 1411 and 1413 are shown. In the embodiment of FIG. 3, each of the plants 1420 is held on a plant advancer 1419 allowing the roots to be immersed in the solution below them.

In one embodiment, plant advancer 1419 includes a track or screen conveyer for holding plants 1420 above a hydroponic solution such that the roots are immersed in the solution. Plant advancer 1419 moves seedling plants 1421 in row 1411 toward center side 1501 of bed 1100. Also illustrated in FIG. 3, plant advancer 1419 also moves seedling 1421 of row 1413 toward far side 1503 of plant bed 1100, in a direction opposite that of row 1411. Therefore, according to this embodiment, when mature plants of row 1413 end up at the far side 1503 of bed 1100 while the mature plant of row 1411 ends up at the center side 1501 of bed 1100.

In the embodiment of FIG. 3, plants 1420 are differentiated into small seedling plants 1421 and large mature plants 1423. According to this embodiment, new seedling plants 1421 are placed in row 1413 at a center side 1501 of bed 1100. Similarly, new seedlings plants 1421 are added to row 1411 at the far side 1503. Since it takes approximately 120 days for the plants to mature, rows 1411 and 1413 can each be designed to hold 120 plants 1420. Therefore, one seedling plant 1421 can be added each day to row 1413, with the row 1413 moving one position toward the far side 1503.

Similarly, a seedling plant 1421 can be added to row 1411 and the row can be moved one position toward center side 1501. This may continue until the rows are filled. This setup is possible since the plants are moveable. In one embodiment, where plant bed 1100 is operational and full of plants, as a seedling is added to the plant bed (e.g., to a row), a mature plant 1423 is harvested. In this embodiment, planting of seedlings progresses continually as plants advance continually along alternating rows advancing in opposite directions toward HPD 2000.

As can be seen, such an embodiment spreads out the harvest into a substantially continuous process. One benefit of this configuration is to permit large full grown, mature plants 1423 to be adjacent to the small seedling plants 1421, allowing the efficient packing of plants, and an efficient use of space. In one embodiment, rows 1410 may also move in the same direction. Additional continuous growth embodiments are discussed below.

Rapid Continual Harvesting

One benefit of the present invention is to accommodate the harvesting of biological material throughout the year, not just in batch growing seasons and to provide the harvesting equipment proximate the biofuel production equipment and the biological material.

In the spirit of simplicity, detailed explanations of one embodiment of the harvesting and processing system and methods will be described in terms of one row and plant 1420. It should be understood that the process may be repeated or conducted simultaneously at multiple areas within system 1000 and the concepts of harvesting and processing can be applied equally to other embodiments and configuration of system 1000 and in particular plant bed 1100.

Referring back to FIG. 2, system 1000 includes HPD 2000. In one embodiment HPD 2000 is self-propelled. In one embodiment, HPD 2000 is run on a track. In the embodiment of FIG. 2, HPD 2000 is directed across rows 1410 to cut and process plants 1420.

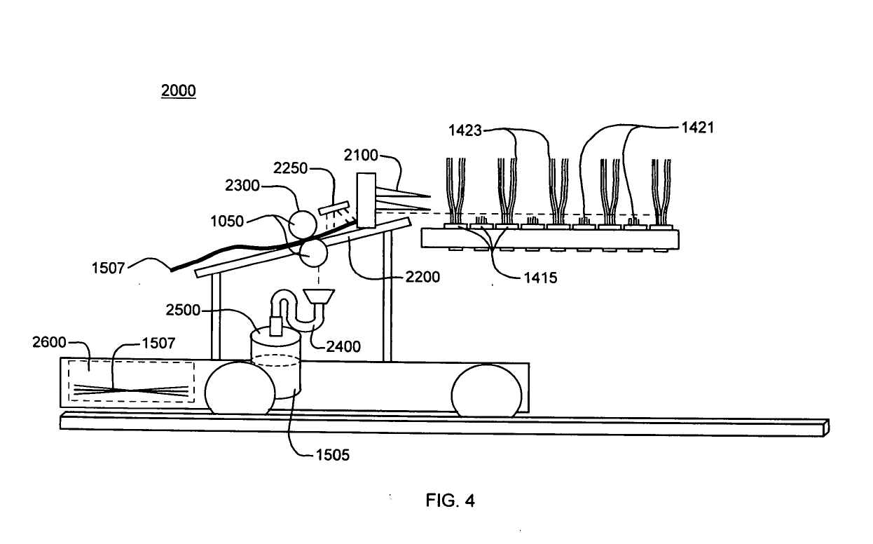

In FIG. 4, one embodiment of the HPD 2000 is shown in an enlarged more detailed view. In the embodiment of FIG. 4, HPD 2000 has cutting section 2100, feeder section 2200 with an optional sprayer 2250, crusher 2300, liquid collection device 2400 with an optional air lock and container section 2500. In one embodiment, HPD 2000 is a single piece of equipment. In another embodiment, HPD 2000 is multiple pieces of equipment.

In one embodiment, cutting device 2100 is configured to cut plants 1420. Cutting device 2100 can be known harvesting equipment, such as the cutting head of a corn stalk harvester as a stem or brush cutter. In one embodiment, HPD 2000 includes a plurality of cutting devices 2100 that are preferably configured to operate simultaneously to increase the rate of harvesting.

In the embodiment of FIG. 4, after plants 1420 have been cut, HPD 2000 is configured to cause plants 1420 to fall onto feeder section 2200. In one embodiment, feeder section 2200 is configured to feed cut plants 1420 to crusher 2300. In one embodiment, feeder section 2200 may be known harvesting equipment. In the embodiment of FIG. 4, feeder section 2200 includes a conveyer (e.g., a belt conveyor or rollers).

In an optional embodiment, sprayer 2250 may be included to spray the cut plants. In one embodiment, sprayer 2250 sprays decontaminant (e.g., water, sulfuric acid) on the cut plants 1420 to prevent deterioration of the sugars in the harvested plant 1420.

In one embodiment, HPD 2000 includes crusher 2300. Crusher 2300 is preferably configured to press juice 1505 from plants 1420. In one embodiment, crusher 2300 includes rollers 1050 for physically pressing the juice 1505 out of plants 1420. In one embodiment, crusher 2300 mills the plant 1420 using means common in the art to extract sugar-containing juice 1505. In one embodiment crusher 2300 may include a two, three or multiple roller mill.

Solid output of the crusher 2300 is commonly known as bagasse 1507. Bagasse 1507 is preferably collected in bulk collection container 2600. In the embodiment of FIG. 4, bulk collection container 2600 is positioned opposite feeding section 2200 on the other side of the crusher 2300. In one embodiment, bagasse 1507 is placed in a bulk collection device 2600 to be unloaded later. As described in more detail below, bagasse is preferably collected and converted into heat and energy for use in system 1000. In one embodiment, bagasse 1507 is conveyed manually, automatically or by another means to be burned in heater 1300 (See FIG. 2).

In one embodiment, HPD 2000 also includes collection device 2400. In one embodiment, juice 1505 from the plants 1420 (e.g., juice that is rich in sugars) is collected into liquid collection device 2400. In one embodiment, an air lock is employed to allow the juice 1505 to enter the container 2500 without introducing air.

In a further embodiment, liquid collection device 2400 is connected to container 2500 and passes the collected juice 1505 to container 2500. In one embodiment, container 2500 includes an air lock, sometimes called a fermentation lock, which allows gases to escape the container 2500, but not allow other gases to enter the container 2500. In one embodiment, the air lock includes an “S” shaped liquid trap. In one embodiment, container 2500 is flooded with an inert gas, or employs a partial vacuum to minimize oxygen in the system which reduces fermentation. This oxygen depleted and sterile environment is preferably sustained in order to promote fermentation, but to inhibit ‘souring’ or contamination of the sugars.

The juice 1505, in another embodiment, is routed through a pipe or hose to a continuous fermentation device, described below.

Therefore, it can be seen that the system employs automatic equipment which quickly harvests, crushes, separates the juice from the bagasse 1507 continuously and rapidly to maximize the carbohydrates (e.g., sugars) collected in container 2500.

Fermentation

In a preferred embodiment, fermentation occurs in fermentation device 130. In one embodiment, containers 2500 are configured to use activated yeast to convert sugars into ethanol in an anaerobic environment. The containers 2500 are preferably sterilized prior to use and may either be filled with an inert gas such as nitrogen, or evacuated. Preferably, the liquids are introduced without introducing oxygen or microbes. Various known air locks or fermentation locks may be employed.

In one embodiment, containers 2500 are removable containers. In one such embodiment, when containers 2500 contain the desired amount of juice 1505 (e.g., containers 2500 are filed or there are no more mature plants 1420, the container is removed and preferably placed in the proper temperature for optimum fermentation. Another container 2500 is added to HPD 2000, and the process continues.

In an alternative embodiment, the juices could be routed through line 2560 in a continuous process. In one embodiment, juices flow through line 2560 to a continuous fermentation unit (CFU) 2550 (both shown in phantom in FIG. 2). In one embodiment, CFU 2550 accepts a continuous flow of juice which moves through a long chamber and ferments as it moves. The internal chamber of CFU 2550 may be a coiled or folded elongated tube. One embodiment of such a continuous fermentation process is described in U.S. Pat. No. 4,357,424 “Process for the Continuous Production of Fermentation Alcohol” by Bu'Lock, Nov. 2, 1982 which is hereby incorporated by reference in its entirety herein.

In one embodiment, containers 2500 are monitored to determine the optimum fermentation. In one embodiment, fermentation takes approximately 72 hours. Following completion of the fermentation stage, solids are removed from the fermented liquid in the container 2500.

In one embodiment, enzymes are introduced to the liquid to enhance fermentation. Exemplary enzymes include protease which is preferably introduced into the fermentation vessel (e.g., container 2500, line 2560). In one embodiment, enzyme is applied to biological material as it is processed by HPD 2000. For example, enzymes may be applied to stalks immediately before, during or immediately after the stalks are crushed. In one embodiment, enzymes are applied to the expressed juices after the stalks are crushed. In a preferred embodiment, glucoamylase enzyme is applied to the extracted juices to optimize glucose concentration for fermentation. In one embodiment, enzymes are selected to promote rapid glucose generation. In another embodiment, enzymes are applied in concentrated form. In another embodiment, enzymes are selected to promote broader operational flexibility and reduce risk of infection. In one embodiment, the selected enzyme includes Novozymes Invertase® and/or Invertase enzyme. Other enzymes may also include Novozymes Alcalase®, Novozymes Spirizyme®, SAN Extra® and Glucoamylase Enzymes. In the preferred embodiment, a sucrase enzyme such as invertase is selected for use in the system.

In one embodiment, microbes are automatically introduced to enhance fermentation. In one embodiment, microbes may be bacteria or yeast. In one embodiment, microbes are injected, pumped as a liquid or introduced as in powder form into fermentation device 130. In one embodiment, the bacterium includes Clostridium acetobutylicum (i.e., Weizmann organisms). In one embodiment, the yeast includes Saccharomyces cerevisiae (i.e., brewer's yeast). In one embodiment, yeast fermentation produces ethanol. In one embodiment, bacteria fermentation produces butanol. In one embodiment, fermentation device 130 includes a batch fermentation device. In another embodiment, fermentation device 130 includes a continuous fermentation device. In one embodiment, fermentation device includes an anaerobic, sterile vessel.

Distillation

Referring back to FIG. 2, the contents of container 2500 are distilled by a distillation unit 2700 which uses heat (e.g., from heater 1300) to concentrate the biofuel (e.g., ethanol, butanol) . Standard known distillation techniques may be implemented. In one embodiment, distillation unit 2700 includes a distillation unit sold by Vendome Copper & Brass Works, Inc. In one embodiment, distillation unit 2700 includes a 2000 gallon unit or larger. In one embodiment distillation unit 2700 includes a continuous distillation unit.

Closed Loop System

In one embodiment, system 1000 is configured to use many of its byproducts. For example, as described above CO₂ which is produced during fermentation is fed back into the greenhouse 100 to stimulate plant growth. Also, bagasse 1507 that is produced during harvesting and processing is recycled to produce heat which is returned to system 1000 (e.g., to greenhouse 100, to distillation unit 2700) and/or to produce energy that can be used to power system 1000.

Alternatively, a co-generation unit 2800 may be employed to burn various materials including coal and bagasse 1507 to produce heat, distil the ethanol and to create electricity. Solar panels (1080 of FIG. 2) may also be used to generate primary or supplementary power in remote areas to provide necessary electrical power for the system 1000.

In one embodiment, bagasse 1507 is burned to heat the greenhouse in heater 1300 (FIG. 2). Also, bagasse 1507 may be burned to produce heat provided to distillation unit 2700 to distil the fermented liquid.

FIG. 1 illustrates three approaches of recycling bagasse 1507. In one embodiment, one or more such approaches are used to produce heat and energy for use in system 1000. In one embodiment, bagasse may be feed directly to boiler 150 to produce steam. In one embodiment, steam is routed to system 1000 using known techniques, to introduce heat to system 1000. Steam produced from boiler 150 may also be directed to a steam turbine (not shown) to produce energy for the operation of system 1000.

In another embodiment, bagasse is formed into pellets in pelletizer 170. The pellets are then burned in furnace 160. In one embodiment, furnace 160 includes a boiler 150 with a steam engine or steam generator. Heat and energy from furnace 160 is then returned to system 1000. In one embodiment, pelletized bagasse is combusted to produce electricity and heat through a biomass generator. In one embodiment, a biomass generator such as that sold by Community Power Corporation (http://www.gocpc.com/).

In another embodiment, bagasse is feed through gasification unit 180 and processed through such processes as Fischer-Tropsch or fast pyrolysis to create synthetic gas for heat/power generation). In one embodiment, gasification unit 180 produces an oil substitute such as BioOil® “Bio-oil” using fast pyrolysis and synthesized jet fuel (e.g., S8FT) using Fischer-Tropsch process. The process of producing such oil substrates may be similar to that used by Dynamotive Energy Systems Corporation or Ensyn Corporation of Wilmington, Del. Gasification unit 180 may be configured to produce heat for use in greenhouse 100. In one embodiment, CO₂ produced by gasification unit 180 is also returned to greenhouse 100.

Alternative Harvesting Embodiments

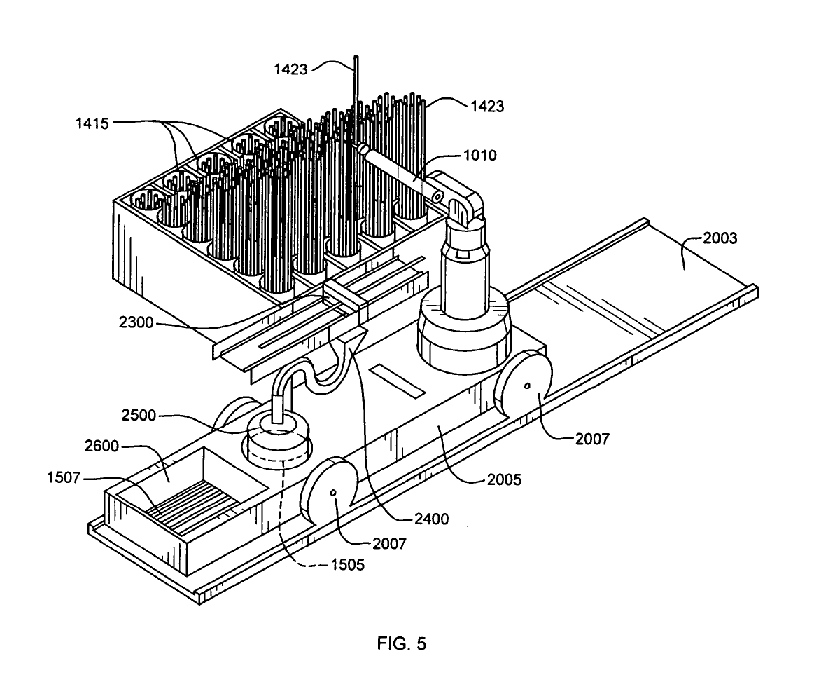

In FIG. 5, is a perspective view of one embodiment of HPD 2000. In FIG. 5, HPD 2000 includes platform 2005 on wheels 2007 riding down track 2003. It also employs one or more robotic arms 1010 in place of (or in some embodiments, cooperating with) cutting device 2100. Robotic arm 1010 may be advanced manually, automatically, or by other means. Robotic arm 1010 grabs the plant by the stalk, pulls the plant 1420 out, and places it in a feeding section 2200. Robotic arm 1010 may include an apparatus for the removal of leaves from the plant 1420 prior to depositing it on a feeding section 2200. In one embodiment, feeding section 2200 advances the plants 1420 toward the crusher 2300.

In another alternative embodiment, HPD 2000 may exist in fixed locations and an automated mechanism brings the harvested plants to HPD 2000 for processing.

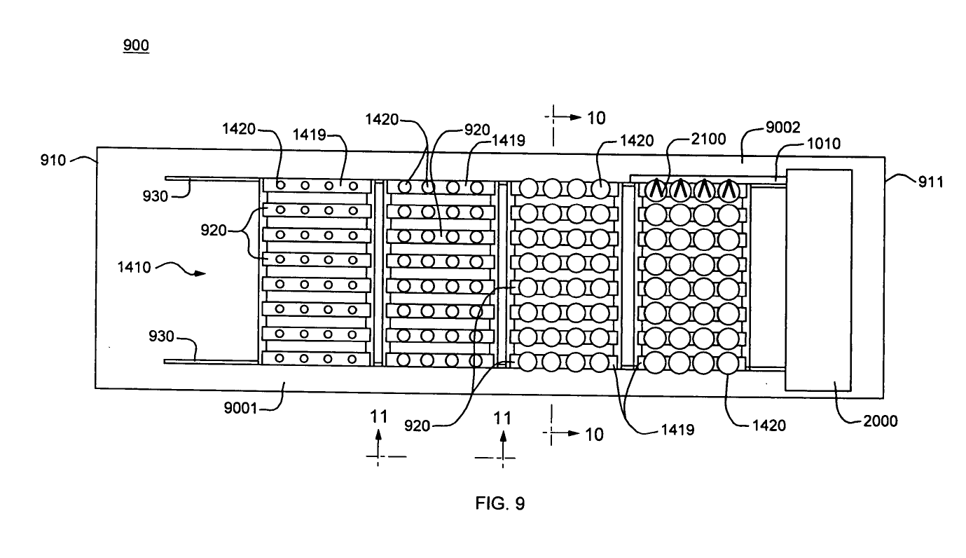

In another embodiment, such as the embodiment illustrated in FIG. 9, HPD 2000 includes a plurality of cutting devices 2100 configured to remove at least a portion of one or more plants from at least two plant levels simultaneously. In one embodiment, HPD 2000 includes a robotic arm configured to control cutting devices 2100.

Alternative Geometries

In the embodiment of FIG. 6, the beds 3005 are circular shaped. In this embodiment, beds 1100 include a plurality of rows 1410 which start at a center location 3001 and radiate to the perimeter 3003. Seeding plants 1421 are planted at the center location 3001. Since the seedlings 1421 are small, they do not require much space.

As they grow, seedlings 1421 are moved toward the perimeter 3003. The outward movement provides each plant 1420 more space. Therefore, there is a near constant plant density across the circular bed 1100 as the plants grow and move outward.

In FIG. 7, another circular geometry is shown for the beds 1100. In this geometry, rows 1410 are dispersed in a spiral fashion starting from a center point 4001 and spiraling to the perimeter 4003. Again, this is employed to provide greater space for the mature plants 1423 as they move outward. Therefore the spiral shape has greater spacing between adjacent rows 1410 as they move outward, based upon their growth at that point. Therefore adjacent points 4005 and 4007 are closer to each other than points 4007 and 4009. Their spacing is based upon the expected size of the plants when they are at that stage of growth. For example, if the plant is at point 4005 in 30 days, point 4007 in 45 days and point 4009 in 52 days, the spacing should match their expected sizes at these stages of growth. In one embodiment, this optimizes the space used providing greater production per square foot of space. There may be one or more separate independent rows 1410 wrapped around each other in each spiral bed 1100.

Vertical Farming

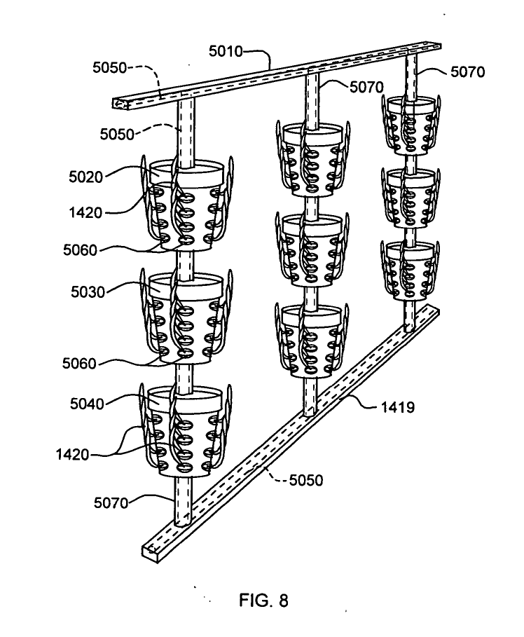

FIG. 8 is illustrates a perspective view of a vertical planting system according to another embodiment of the present invention. (A single vertical system is shown in detail here, however, it is to be understood that there are a plurality of vertical systems in each bed 1100.) In the embodiment of FIG. 8, planter pots 5020, 5030, 5040 are held vertically by supports 5070. Supports 5070 are held and moved by plant advancer 1419. As stated above, plant advancer may be made up of conveyors, belts, rollers, linear actuators capable of moving the vertical planter pots 5020 along the track.

In one embodiment, each planter pot 5020 has a plurality of plants 1420 growing out of a plurality of openings 5060 in the planter pots 5020, 5030, 5040. The roots of plants 1420 are inside of planter pots 5020, 5030, 5040. A fluid supply 5050 runs through the supports 5070 to provide water and necessary nutrients to the roots of plants 1420. These vertical planters may be the commercially available products such as the products hydrostacker.com or vertigro.com.

In one embodiment, a fluid supply 5050 provides water and other nutrients to the inside of planter pots 5020, 5030, 5040. The planter pots may hold a supply of this fluid and grow plants 1420 hydroponically. Alternatively, there may be soil in planter pots 5020, 5030, 5040 and the plants 1420 may be grown conventionally. And in still another embodiment, fluids will be sprayed intermittently on the roots of plant 1420 to grow them aeroponically. The planter pots may be allowed to leak the fluid out of the bottom to the next lower planter pot to keep the fluids circulating. The vertical embodiments no longer require a fluid bed as does the single layer system.

Due to the more complicated geometry, harvesting of this multi-level system may require use of the programmable robotic arm (1010 of FIG. 5). The planter pots 5020, 5030, and 5040 may be moved vertically and rotated to expose the plants 1420 to the robotic arm 1010 for harvesting.

In an alternative embodiment, an overhead track 5010 may be added for stability in holding the vertical planters 5020, 5030, 5040, or for providing the necessary fluids to the plants 1420. Also, the planter pots 5020, 5030, 5040 may hang from overhead track 5010. In one embodiment, where vertical planters 5020, 5030 are configured to support plants growing in lower vertical planters 5030 and 5040 respectively.

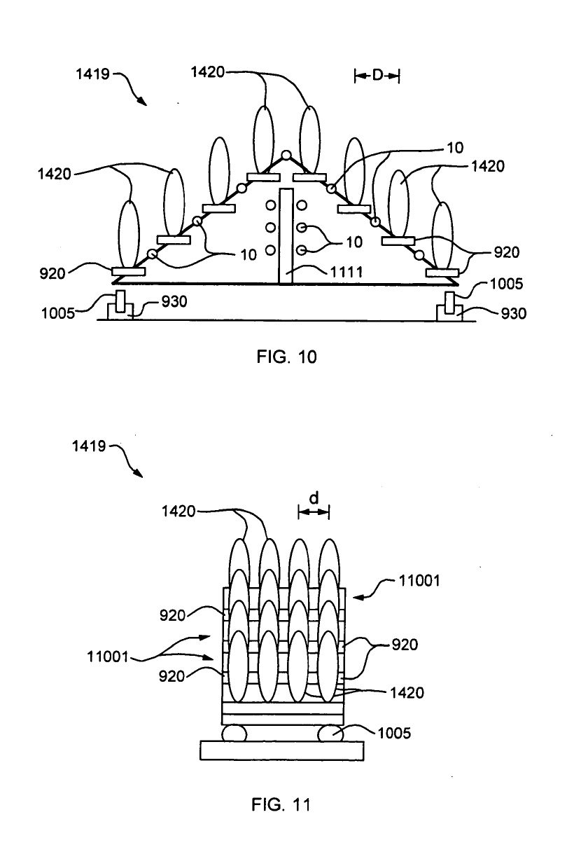

FIGS. 9-11 illustrates one embodiment of a vertical farming system 900 of the present invention. The vertical farming system 900 includes a first end 910, a second end 911, HPD 2000 located proximate the second end, a plurality of plant advancers 1419 having a plurality of plants 1420. In one embodiment, plant advancers 1419 are moveable from a first position 9001 to a second position 9002 wherein the first position is proximate first end 910 and the second position is proximate second end 911. In the embodiment illustrated in FIG. 9, plant advancers 1419 are aligned in row 1410 and HPD 2000 is located at one end of row 1410 proximate the second end 911. In one embodiment, an advancer guide 930 cooperates with plant advancers 1419. In one embodiment, advancer guide 930 is configured to guide plant advancer 1419 from first position 9001 to second position 9002. In one embodiment, illustrated in FIGS. 9 and 10, advancer guide 930 includes a track that cooperates with wheels 1005 on plant advancer 1419 and guides plant advancers 1419 as they move from first position 9001 to second position 9002. In other embodiments, advancer guides may include conveyors, chain drives, belt drives, linear actuators and the like.

In operation, plant advancers 1419 are moved from first position 9001 to second position 9002 as plants 1420 mature. When plants 1420 are ready for harvesting, HPD 2000 is operated to harvest plants 1420 and process the plants as described herein. In one embodiment, as all of the plants 1420 on the plant advancer 1419 are harvested, plant advancer 1419 may be removed from second position 9002 to allow the next plant advancer 1419 in line to move into second position 9002. The removed plant advancer 1419 may then be returned to first position 9001 to accept less mature plants (e.g., seedlings) and begin advancement again toward second position 9002. In one embodiment, the floor upon which plant advancers 1419 are supported is pitched toward second position 9002 such that when plant advancer 1419 is removed from row 1410, the remaining plant advancers in the row move by gravity toward second position 9002. In one embodiment, in which plants 1420 includes sweet sorghum, a complete cycle from first position 9001 to second position 9002 is between approximately 90 days and approximately 120 days, preferably between approximately 90 days and approximately 100 days. In one embodiment, the cycle is 120 days.

It should be recognized that a biofuel production system of the present invention could include a multiplicity of rows 1410 having multiplicity of plant advancers 1419 each with a multiplicity of plants 1420 to accommodate a large and efficient production. Moreover, a large production facility might include a plurality of HPD 2000 that are either stationary or mobile relative to plant advancers 1419 and rows 1410.

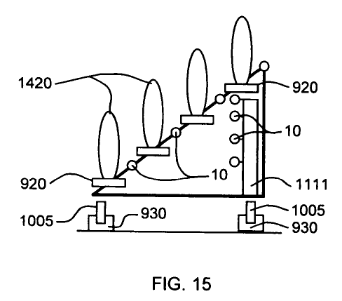

FIG. 10 illustrates a cross section of vertical fanning system 900 and in particular cross section A-A of plant advancer 1419 of vertical farming system 900. FIG. 11 illustrates cross section B-B of plant advancer 1419 of vertical farming system 900. In the embodiment illustrated, plant advancer 1419 is in a pyramid configuration. In an alternative configuration, illustrated in FIG. 15, plant advancer 1419 is in a right triangle configuration. As illustrated, in one embodiment, plant advancer 1419 includes a plurality of plants 1420 configured to grow in a staggered vertical configuration. In one embodiment, plants are arranged in rows 11001 on plant advancer 1419. In one embodiment the rows 11001 correspond with troughs 920. In one embodiment, plants are densely arranged. In one embodiment, plants are spaced a distance d of two inches apart. In one embodiment, plants are spaced a distance of between approximately two and approximately four inches apart. In one embodiment, plants are spaced a distance d between approximately three inches and approximately four inches apart.

In one embodiment, plants 1420 are also held in plant advancer 1419 such that the roots of plants 1420 are located within troughs 920. In one embodiment a central fluid distribution system (not shown) provides a nutrient feed source that flows through troughs 920 to facility the hydroponic growth of plants 1420 as disclosed herein. In another embodiment, troughs 920 include sprayers that spray nutrient rich solution on the roots of plants 1420 to promote aeroponic growth of plants 1420.

In one embodiment, troughs 920 are arranged in an off-set vertical configuration. In one embodiment, an advantage of the offset vertical configuration is the ability to increase the density of plants (e.g., preferably sweet sorghum) in both a horizontal and vertical directions. Thus in the embodiment illustrated in FIGS. 9-11, troughs 920 are arranged parallel to one another with adjacent troughs being at different elevation. In one embodiment, best illustrated in FIG. 10, troughs 920 are arranged in a pyramid configuration. In one embodiment, the pyramid configuration positions troughs 920 in an overlapping stacked configuration such that the distance D between plants in adjacent rows 1420 is small. In one embodiment, the distance D between plants 1420 (e.g., the distance between stalks of sweet sorghum) in adjacent rows is between approximately two inches and approximately six inches. In one embodiment, the distance D is approximately three to approximately four inches. In one embodiment the distance D between adjacent rows is approximately the same as the distance d between plants in the same row.

In one embodiment, the pyramid configuration is an open pyramid configuration that allows for the introduction of light from within the pyramid structure to illuminate the desired portion of plants 1420. In a preferred embodiment light is shone on multiple sides of plants 1420. In one embodiment, light is shone on multiple sides of panicle of plants 1420. In one embodiment, light source 10 provides light to plant 1420. In one embodiment light source 10 supplements ambient light that plant 1420 receives through greenhouse 100. In one embodiment, light source 10 is an LED light. In one embodiment, an LED light source is preferred because it produces very little heat and consumes relatively little power. Thus a multiplicity of LEDs can be placed in close proximity to plants 1420 such that plants 1420 receive substantially constant light of a substantially uniform intensity on multiple sides of plant 1420. The use of light source 1420 in connection with a vertical planting configuration also permits plants (e.g., Sweet Sorghum) to be planted very close to each other and in a close vertical configuration. Whereas traditional farming might preclude such a configuration, the use of supplemental light source 10 and in particular LED light source, facilitates exposure of densely arranged plants to much needed light. In one embodiment, an example of which is shown in FIGS. 10 and 15, light source 10 is positioned throughout plant advancer 1419. In one embodiment, light stand 1111 is positioned within plant advancer 1419. In one embodiment light stand 1111 includes a plurality of light sources 10 that are configured to illuminate plants 1420 through an open structure of plant advancer 1419.

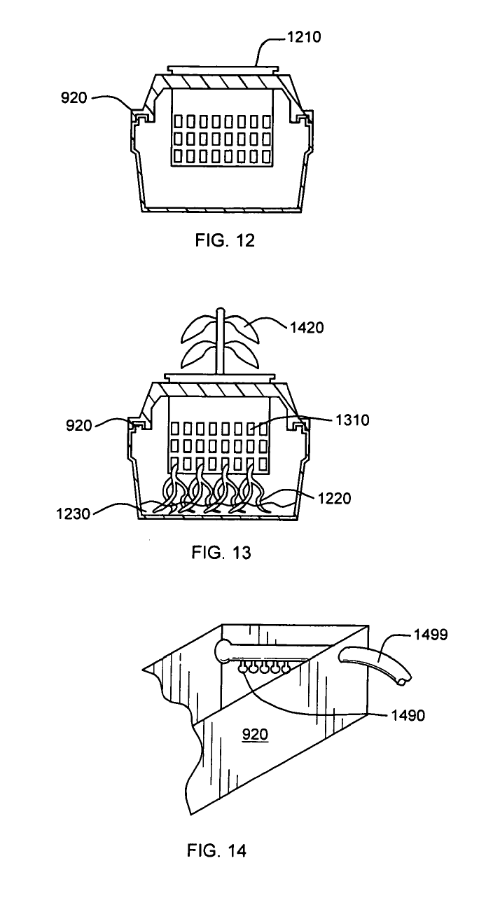

FIG. 12 illustrates a cross section of one embodiment of trough 920. In one embodiment, trough 920 includes puck 1210. In one embodiment, puck 1210 includes a plurality of apertures through which the roots of plants 1420 may protrude. In one embodiment, each plant 1420 is grown in a puck 1210. When plant 1420 is harvested, the old puck 1420 is removed from plant advancer 1419. In one embodiment, a new puck 1420 with a seedling is replaced in trough 920 when plant advancer 1419 is placed in first position 9001.

FIG. 13 illustrates a cross section of one embodiment of trough 920. In one embodiment, roots 1220 of plants 1420 protrude through apertures 1310 and are fed by nutrients 1230 flowing through trough 920. In one embodiment, illustrated in FIG. 14, trough 920 is fed by nutrient line 1499. In one embodiment, misters 1490 apply nutrient rich liquid into trough 920. In one embodiment, nutrient rich liquid cascades through troughs 920 that are located higher on plant advancer 1419 to troughs 920 that are lower on plant advancer 1419. In another embodiment, one nutrient feed line is associated with each trough 920 and after the fluid flows through that trough the liquid is recycled through a drain to a central point.

While the invention has been described above with respect to particular embodiments, modifications and substitutions within the spirit and scope of the invention will be apparent to those of skill in the art. It should also be apparent that individual elements identified herein as belonging to a particular embodiment, may be included in other embodiments of the invention.

1-47. (canceled)

48. A substantially self-contained biofuel production system comprising:
an enclosure having a controlled environment;
a processing device provided substantially within the enclosure configured to harvest biological material within the enclosure as the biological material matures and to extract liquid from the harvested biological material; and
a fermenting device fluidly connected to the processing device and configured to accept and ferment the liquid from the processing device.

49. The biofuel production system of claim 48 wherein, the processing device expresses bagasse and the biofuel production system further comprises a heat return system configured to process the bagasse to produce heat and deliver the heat to the enclosure.

50. The biofuel production system of claim 49, further comprising:
a power generator configured to convert the produced heat to power; and
a power return system configured to return at least a portion of the power produced by the power generator to the biofuel production system.

51. The biofuel production system of claim 48, wherein the processing device expresses bagasse and the biofuel production system further comprises a combustion device proximate the at least one processing device that is configured to combust the bagasse and to produce at least one of heat and power for recycling in the biofuel production system.

52. The biofuel production system of claim 48, further comprising:
a distillation device proximate the fermenting device and configured to receive fermented liquid from the distillation device and distill the fermented liquid.

53. The biofuel production system of claim 48, wherein the biological material includes sweet sorghum.

54. The biofuel production system of claim 48, wherein the processing device includes:
a cutting device configured to cut the biological material;
a feeding section configured to catch the biological material;
a sprayer configured to spray liquid on the cut biological material;
a crusher configured to extract liquid from the cut biological material; and
a liquid collection device configured to collect the extracted liquid.

55. The biofuel production system of claim 48, further comprising:
a gasification unit operating in at least one of a Fischer-Tropsch and a pyrolysis process, the gasification unit being proximate the processing device and configured to gasify bagasse produced from the processing device and configured to produce heat and biofuel.

56. The biofuel production system of claim 48, wherein the fermenting device is configured to directly receive the extracted liquid from the processing device.

57. The biofuel production system of claim 48, further comprising:
a line fluidly connecting the processing device and the fermenting device and configured to transport the liquid from the processing device to the fermenting device.

58. A biofuel production method comprising:
producing plants in a controlled environment enclosure;
harvesting the plants within the controlled environment enclosure;
extracting liquid from the harvested plants using a processing device located within the controlled environment enclosure;
transporting the extracted liquids to a fermenter in fluid connection with the processing device;
fermenting the extracted liquids in the fermenter; and
distilling the fermented liquids to product biofuel.

59. The method of claim 58 further comprising:
introducing at least one of enzymes and microbes to the extracted liquid to enhance fermentation.
